(19) United States
(12) Patent Application Publication
(10) Pub. No.: US 2008/0279830 A1
(43) Pub. Date: Nov. 13, 2008

(54) SPONGY EPITHELIAL CELL SCAFFOLD FOR VASCULARIZING WOUNDS

(75) Inventor: WARREN K. HOEFFLER, Mill Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Xgene Corporation, Sausalito, CA (US)

(21) Appl. No.: 12/117,516

(22) Filed: May 8, 2008

Related U.S. Application Data
(60) Provisional application No. 60/916,756, filed on May 8, 2007.

(51) Int. Cl. A61K 35/36; C12N 5/08; A61P 17/02

(52) U.S. Cl. 424/93.7; 435/371

(57) ABSTRACT

The present invention provides isolated apoptotic epithelial scaffolds. The scaffolds find use in promoting re-vascularization of wounded tissues. The present invention also provides isolated or ex vivo tri-layered cell sorted tissues comprised of a bottom layer comprised of an apoptotic epithelial scaffold, a middle layer comprised of mesenchymal cells, and a top layer comprised of non-differentiated epithelial cells. The invention further provides in vitro methods for generating isolated or ex vivo tri-layered cell sorted tissues by exposing cultured mixtures of epithelial and mesenchymal cells to an inflammatory cytokine. Further provided are methods of promoting wound healing and re-vascularization of wounds by contacting wounded tissue with the apoptotic epithelial scaffolds of the invention.

### CROSS-REFERENCES TO RELATED APPLICATIONS

The present application claims the benefit of U.S. Provisional Application No. 60/916,756, filed May 8, 2007, the entire disclosure of which is hereby incorporated herein by reference.

### FIELD OF THE INVENTION

The present invention is directed to the creation of a spongy scaffold composed of apoptotic epithelial cells. The apoptotic epithelial cell scaffolds find use in the vascularization at a wound site. The epithelial cell scaffold is generated in vitro, i.e., in tissue culture, by the sorting of cells from a homogeneous cell mixture into discrete layers and requires that the epithelial cells respond to the wound conditions of physical cell disruption, inflammatory cytokines, and serum. The invention further provides isolated or ex vivo tri- layered cell sorted tissues comprised of a bottom layer comprised of an apoptotic epithelial scaffold, a middle layer comprised of mesenchymal cells, and a top layer comprised of non- differentiated epithelial cells. The invention further provides in vitro methods for generating isolated or ex vivo cell sorted tissues comprising spongy epithelial scaffolds comprised of apoptotic epithelial cells by exposing cultured mixtures of epithelial and mesenchymal cells to an inflammatory cytokine. The spongy scaffold and tri-layered cell sorted tissues are suitable for placement into wound beds to facilitate healing. The invention also provides methods of repairing wounded tissue using the in vitro generated tissues described herein.

### BACKGROUND OF THE INVENTION

There has been a large disparity between the success rate of grafting tissue using in vivo created tissues versus tissues that are grown using in vitro tissue culture techniques. This trend is illustrated in the area of skin grafting, which is probably the most often grafted tissue and also has the most commercial ventures attempting to supply the marketplace with state of the art skin grafts grown using tissue culture techniques.

The problem has been that the living cells contained in the grafts do not persist after grafting. Thus, Dermagraft™ is a polylactic material upon which autologous fibroblasts are seeded, but the product has not fulfilled the market need, leading to Advanced Tissue Sciences going out of business and Smith and Nephew dropping the product. The main problem was that the cells that were grafted did not persist in the wound bed, rather they died within a few days.

Likewise, Apligraft™ (Graftskin™) produced by Organogenesis contained both allogeneic keratinocytes and fibroblasts, but again the cells died off in the wound bed and no cells survived to become part of the replacement cells. The cells showed marked loss of viability by day 4 in culture (Griffiths, et al., Tissue Eng. (2004) 10(7-8):1180-1195).

The declining viability of the grafted cells is most likely due to the lack of a vascular network. The constructs have no vasculature to link into in the wound bed. In contrast, when the body's skin is removed from one location and grafted onto a second location the take rate is high (80-95%) and the tissue re-vascularizes because the graft already has a vascular network. Including a premade vascular network in an in vitro made skin is technically difficult because in the absence of blood flow newly formed vessels degrade. A better solution is to provide an intermediate structure that would facilitate infiltration of blood vessel forming cells from the patient into the graft.

The most powerful inducer of blood vessels is Vascular Endothelial Growth Factor (“VEGF”), which when supplied as a protein (Post, et al., Cardiovasc Res (2001) 49(3):522-531) or a gene (Rutanen, et al, Curr Cardiol Rep (2001) 3(1):29-36; and Lee, et al., Ann Thorac Surg (2000) 69(1): 14-23) stimulates endothelial cells to form capillary networks. VEGF has been directly applied to patient wounds but has not worked well in clinical trials (Henry, et al., Circulation (2003) 107(10):1359-1365; and Henry, et al. Am Heart J (2001) 142(5):872-880). Although VEGF is a key factor it may not often be the limiting factor in a wound. The creation of the right wound environment where cells are able to interact as they normally do in a wound would allow cells to make VEGF and the other needed cytokines required for vascularization.

U.S. Pat. No. 6,815,202 describes the use of spontaneous cell sorting to recreate epithelial/mesenchymal tissue layers using a mixed cell slurry, and relying upon the cells to spontaneously cell sort to recreate tissue layers. This product uses superior technology because the placement of cells achieved is determined by cell migrations of the mixed pool of cells, allowing true cell signaling to occur between cells (See also, Wang, et al., J Invest Dermatol (2000) 114:674-680; Funk, et al., Experimental Cell Research (2000) 258(2):270-278; Chen, et al., Nature Genetics, (2002) 32:670-675; and Hachiya, et al., J Invest Dermatol (2005) 125:364-372).

All current models lack the ability to re-vascularize, but all models are also based on normal undamaged skin. Graftable tissue made in vitro is needed that recapitulates early steps in wound healing that will attract the patients' cells into the graft for vascularization. A graftable construct is needed that when placed into a wound will facilitate vascularization, promoting wound healing. The present invention addresses this and other needs.

### BRIEF SUMMARY OF THE INVENTION

The re-vascularization of tissues is a key step in their successful grafting onto patients. The present invention is based, in part, on the observation that epithelial cells when triggered to respond to wound conditions will construct an early provisional matrix, or scaffold that is a first step in re-vascularization of a wound. Invasion of blood cell types, including endothelial cells, and macrophages (or pluripotent hemangioblasts), and mesenchymal cells (e.g., fibroblasts), into the apoptotic epithelial cell scaffold facilitates the establishment of actual vasculature. The infiltrating cells can either be cultured with the scaffold ahead of time, or the scaffold can be placed on the wound and patient blood cells types can then populate the scaffold. By deliberately creating the scaffold, accelerated wound healing results, with better ‘take rates’ and reduced scarring.

The invention is based, in part, on the creation and use of a spongy apoptotic (i.e., made of dead cells) scaffold produced by differentiated epithelial cells for the purpose of re-vascularizing a wound. Epithelial cells normally terminally differentiate as they migrate to the outer tissue layers and in the case of skin apoptose to form dead squames residing on the outer skin surface. These dead cells provide a crucial function in the creation of squames that are lipid encased sacks of tough non-soluble proteins that serve as a barrier to invading organisms from the outside world while also keeping water in the body. During wound healing a distinct pathway for epithelial cells is proposed where a different set of structural proteins and cell surface proteins are expressed, but that also leads to terminal differentiation and apoptosis. These apoptotic epithelial cells provide the framework for reestablishing a vascular network at the wound site, a second unique function just as important as the use of apoptotic epithelial in creating the outer skin barrier.

The present invention observes the presence of this scaffold as an isolated static entity produced in vitro using tissue culture cells. By including epithelial cells that were at least partially differentiated, controlling exposure of the cells to serum, and exposing cells to inflammatory cytokines, the epithelial cells will form a spongy scaffold underneath the mesenchymal layers. The structure of the scaffold is distinct because when properly formed it is populated with circular openings of a size ideally suited for containing newly formed capillary networks within their boundaries. The apoptotic epithelial cell scaffold finds use to re-vascularize a wound. Although illustrated here using skin cells, a similar phenomenon could be observed using other combinations of epithelial/mesenchymal cells, including endothelial cells/smooth muscle cells to rebuild blood vessels, lung epithelia and mesenchyme, and gut epithelia and mesenchyme. Since the scaffold is made of apoptotic cells it lends itself to being stored as a non-living matrix that could be used alone or in combination with mesenchyme cell constructs, or epithelial/mesenchymal cell constructs, and therefore facilitate its use in medical applications.

Accordingly, in one aspect, the invention provides an isolated apoptotic epithelial cell scaffold. In some embodiments, the apoptotic epithelial cell scaffold is freeze-dried. The apoptotic epithelial cell scaffolds find use for placement into a wound bed to be occupied by the patients own cells from their blood and surrounding tissues.

In a further aspect, the invention provides an isolated tri-layered cell sorted tissue comprised of

a) a bottom layer comprised of an apoptotic epithelial cell scaffold;

b) a middle layer comprised of mesenchymal cells; and

c) a top layer comprised of non-differentiated epithelial cells.

The cells comprising the apoptotic epithelial cell scaffold are primarily non-viable (i.e., not living). The cells comprising the middle mesenchymal layer and the top undifferentiated epithelial cell layer are primarily viable (i.e., living, capable of dividing).

With respect to the embodiments of the cell sorted tissue, in some embodiments, the epithelial cells are keratinocytes. In some embodiments, the mesenchymal cells are fibroblasts.

In some embodiments, the epithelial cells are from a tissue source selected from the group consisting of skin, kidney, colon, prostate, breast, heart, urogenital tissue, vagina, lung, gut and blood vessels. In some embodiments, the mesenchymal cells are from a tissue source selected from the group consisting of skin, kidney, colon, prostate, breast, heart, urogenital tissue, vagina, lung, gut and blood vessels. In some embodiments, the mesenchymal cells are endothelial cells or smooth muscle cells.

In some embodiments, epithelial cells and mesenchymal cells are present in about equal numbers. In some embodiments, epithelial cells and mesenchymal cells are present in about a 1:1, 2:1 or 3:1 ratio (epithelial cells:mesenchymal cells).

In some embodiments, the tri-layered cell sorted tissue does not comprise a basement membrane zone (BMZ).

In another aspect, the invention provides in vitro methods of generating a tri-layered cell sorted tissue comprising a discrete bottom cell layer, a discrete middle cell layer and a discrete top cell layer. In some embodiments, the methods comprise the steps of:

i) providing a homogenous mixture of cells, said mixture comprising epithelial cells and mesenchymal cells; and

ii) contacting said mixture with a membrane or a connective tissue component in the presence of an inflammatory cytokine and under conditions wherein said mixture spontaneously sorts into said bottom, middle and top discrete cell layers, wherein said discrete bottom layer comprises an apoptotic epithelial cell scaffold, said discrete middle layer comprises mesenchymal cells and said discrete top cell layer comprises non-differentiated epithelial cells.

With respect to the embodiments of the methods for in vitro generation of tri-layer cell sorted tissues, in some embodiments, the epithelial cells are keratinocytes. In some embodiments, the mesenchymal cells are fibroblasts.

In some embodiments, the epithelial cells are from a tissue source selected from the group consisting of skin, kidney, colon, prostate, breast, heart, urogenital tissue, vagina, lung, gut and blood vessels. In some embodiments, the mesenchymal cells are from a tissue source selected from the group consisting of skin, kidney, colon, prostate, breast, heart, urogenital tissue, vagina, lung, gut and blood vessels. In some embodiments, the mesenchymal cells are endothelial cells or smooth muscle cells.

In some embodiments, the homogenous mixture of cells comprises about equal numbers of epithelial cells and mesenchymal cells. In some embodiments, epithelial cells and mesenchymal cells are present in about a 1:1, 2:1, 3:1 ratio (epithelial cells:mesenchymal cells).

In some embodiments, the inflammatory cytokine is selected from the group consisting of IL-1α, IL-1β, IL-6, IL-8, IL-12, IL-18, TNF-α, or a mixture thereof. In some embodiment, the inflammatory cytokine is tumor necrosis factor alpha (TNF-α). In some embodiments, the TNF-α is produced by the mixture of cells. In some embodiments, the TNF-α is exogenously added to the mixture of cells.

In some embodiments, the conditions for sorting comprise at least 1.0 mM Ca²⁺. In some embodiments, the methods further comprise the step of subjecting the epithelial cells and the mesenchymal cells to physical disruption (e.g., trypsinization) before providing them in the homogenous mixture.

In some embodiments, the tri-layered cell sorted tissue does not comprise a basement membrane zone (BMZ). In some embodiments, a basement membrane zone (BMZ) is formed in the tri-layered cell sorted tissue between the middle and top layers and not between the middle and bottom layers.

In a related aspect, the invention provides methods of repairing wounded mesenchymal tissue and the underlying vascular bed. In some embodiments, the methods comprise

i) generating in vitro an isolated tri-layered cell sorted tissue comprised of
a) a bottom layer comprised of an apoptotic epithelial cell scaffold;
b) a middle layer comprised of mesenchymal cells; and
c) a top layer comprised of non-differentiated epithelial cells

ii) contacting the wound bed containing mesenchymal tissue and damaged blood vessels with the bottom layer of the tri-layered cell sorted tissue, wherein the apoptotic epithelial cell scaffold promotes the infiltration of new blood vessels in the wound, thereby repairing wounded mesenchymal tissue and the underlying vascular bed.

In another aspect, the invention provides methods of promoting vascularization of wounded tissue. In some embodiments, the methods comprise

i) generating in vitro an apoptotic epithelial cell scaffold; and

ii) contacting the wound bed containing mesenchymal tissue and damaged blood vessels with the apoptotic epithelial cell scaffold, wherein the apoptotic epithelial cell scaffold promotes the infiltration of new blood vessels in the wound, thereby promoting vascularization of wounded tissue.

In some embodiments, the method comprises contacting the wounded tissue with an apoptotic epithelial cell scaffold.

With respect to the embodiments for repairing wounded tissue, in some embodiments, the tissue is skin. In some embodiments, the epithelial cells are keratinocytes. In some embodiments, the mesenchymal cells are fibroblasts.

In some embodiments, the epithelial cells are from a tissue source selected from the group consisting of skin, kidney, colon, prostate, breast, heart, urogenital tissue, vagina, lung, gut and blood vessels. In some embodiments, the mesenchymal cells are from a tissue source selected from the group consisting of skin, kidney, colon, prostate, breast, heart, urogenital tissue, vagina, lung, gut and blood vessels. In some embodiments, the mesenchymal cells are endothelial cells or smooth muscle cells.

### Definitions

An “apoptotic epithelial cell scaffold” refers to a matrix primarily composed of epithelial cells that have undergone or are undergoing programmed cell death.

A “non-differentiated epithelial cell” or “undifferentiated epithelial cell” interchangeably refers to epithelial cells capable of regenerating the epithelium as is required during re-epithelialization (i. e., capable of cell division).

The term “re-epithelialization” or “re-epithelializing” refers to the activity of epithelial cells that regenerate (i.e., divide) to establish a new or expanded layer or population of epithelial cells.

As used herein, the term “discrete” refers to distinct or clearly distinguishable.

The term “homogenous cell mixture” refers to an unsorted composition of single cells of one cell type (e.g., epithelial cells) or multiple cell types (e.g., epithelial cells and mesenchymal cells).

The term “sort” or “sorted” refers to separation or arrangement of cells from a homogenous cell mixture.

As used herein, the term “bottom” refers to the layer or surface forming attached to the transwell membrane or bottom of the culture dish.

A “cell slurry” refers to a composition comprising a homogenous mixture of cells.

### BRIEF DESCRIPTION OF THE DRAWINGS

FIG. 1A illustrates a cross-section of tissue created by the process described in U.S. Pat. No. 6,815,202. Discrete dermal and epidermal layers are created using the process of spontaneous cell sorting. The photograph is of a H/E (hematoxylin/eosin) stained 6 mm section.

FIG. 1B illustrates the process used to create cell sorted tissue layers, as described in U.S. Pat. No. 6,815,202, where both epithelial cells (for example, keratinocytes from skin) and mesenchymal cells (for example, dermal fibroblasts from the skin) are grown separately in tissue culture, harvested by trypsinization from culture plates, mixed together into a slurry, and then plated into transwell inserts containing a semipermeable membrane at the bottom that allows medium to enter up trough the filter to feed the cells.

FIG. 2 illustrates an hematoxylin-and-eosin (“H/E”) stained thin cross-section of a tri-layer cell sorted tissue created by the present invention. A tissue different from the tissue shown in FIG. 1 is created. Here, the cells perceive that they are in a wound, and are treated to send signals to the cells that they are needed to respond to wound conditions. The differentiated epithelial cells migrate to the bottom of the wound bed, which in the transwell set-up is to the surface of the filter. The differentiated epithelial cells undergo apoptosis and create a provisional matrix scaffold that looks spongy in that there are holes and gaped areas where blood cell types, including macrophages and endothelial cells can begin to occupy space in this spongy scaffold. This figure demonstrates success in re-creating the structure made by cells as a first step in re-vascularization. The initial provisional matrix created by wound induced keratinocytes is attached to the filter and the dermal fibroblasts are on top, superficially upside down compared to normal skin. The very top of the sample shows a thin layer of keratinocytes in the process of re-epithelialization, and is right side up. A standard basement membrane zone (BMZ) only forms between the re-epithelializing keratinocytes and the dermal fibroblasts and never between the dermal fibroblasts and the wound induced keratinocytes at the bottom because these cells are poised to remodel. Note the spongy appearance of the bottom provisional matrix.

FIG. 3 illustrates TUNEL stained keratincytes (apoptotic cells, green) merged with propidium iodide Fb nuclei (non-apoptotic cells, red). FIG. 3 demonstrates that the spongy matrix seen in FIG. 2, above, is made up of apoptotic cells, meaning that these cells are programmed to die, and therefore that the scaffold is made up of dead cells. Keratinocytes are known to migrate into the wound bed, but their fate once there has not been clear. One group speculated that the keratinocytes might apoptose, based on an argument that it is the most energy efficient mechanism for getting rid of them once no longer needed (Greenhalgh, Intl J Biochem Cell Bio (1998) 30:1019-1030). In contrast, FIG. 3 demonstrates that the keratinocytes undergo apoptosis en masse, and importantly, leave behind a scaffold structure. This scaffold is the initial provisional matrix that is the precursor for granulation tissue that will evolve as endothelial cells and fibroblasts begin to take residence within this scaffold structure. FIG. 3 shows that the lower keratinocyte layer specifically lights up (green) with the fluorescent fluorescein labeled tag of the TUNEL assay indicating that it is the keratinocyte layer that is specifically apoptosing, while the propidium iodide labeling (red) of the fibroblast nuclei at the top have few stained cells. Method: The TUNEL assay is conducted by solubilizing the cell membranes with triton X-100, and then incubating with a mixture of fluorescein conjugated-dUTP and terminal deoxynucleotidyl transferase that label 3′ OH ends of DNA cleaved during apoptosis (green)(Promega). The sample was co-stained with propidium iodide (red).

FIG. 4 demonstrates that proteins known to be expressed in wounds are also produced in significant amounts by the cells in the tri-layer cell sorted tissues. Immunohistochemistry using antibodies specific to proteins expressed in wounds, e.g., fibronectin, vimentin, Keratin 16, was used. Keratinocytes are at the bottom, with fibroblasts above them, superficially, the inverse of normal skin. FIG. 4A shows fibronectin stained fibroblasts. Background staining only for keratinocytes. Fibronectin is present in the top half containing the mesenchymal cells (here fibroblasts) stained red. FIG. 4B shows 4′-6-Diamidino-2-phenylindole (DAPI) stained nuclei, blue, merged with above fibronectin staining, illustrating more clearly that the cells at the bottom layers are not making fibronectin. FIG. 4C shows vimentin stained fibroblasts. The fibroblasts at the top half also make vimentin, a typical marker for fibroblasts. FIG. 4D shows Keratin 16 staining of keratinocytes at bottom against the filter. The epithelial cells, here keratinocytes, expressing keratin 16 migrate to the lowest position right on top of the filter. Keratin 16 is a marker for early wound response by keratinocytes. FIG. 4E shows hyaluronic acid staining of both fibroblasts at the top and keratinocytes at the bottom. The complete sample has an abundance of hyaluronic acid (HA), known to be produced by cells that are positioning themselves to remodel and migrate further into position. HA keeps the tissue hydrated and the individual cells more loosely associated or connected. HA binding protein was used similarly to an antibody to obtain this picture, since HA is not a protein and so antibody reagents are difficult to generate.

FIG. 5 illustrates that there is a single cell layer of epithelial cells (e.g., keratinocytes) in the in vitro culture that represents cells that are re-epithelializing the wound. FIG. 5 demonstrates that some of the epithelial cells added to the mixed cell slurry migrate to the very top of the recreated wound because they are involved in re-epithelialization. These cells at the top are more basal in character, including epithelial stem cells. Therefore, the tri-layer cell sorted tissues are not really upside-down; rather there is a spongy layer of apoptotic epithelial cells at the bottom and then mesenchymal layers in the middle, and then again an undifferentiated epithelial layer at the top. A) collagen IV immunostaining shows a line at the top that is the beginning of a reforming basement membrane zone. B) shows the same staining method on a normal foreskin control sample. C) shows that a few epithelial cells at the top are making Ki67 that is a marker for proliferation, and therefore also stem cell function. D) foreskin control stained for Ki67 marker (spots at basement membrane zone stained). FIG. 5 illustrates that although the differentiated epithelial cells migrate to the bottom of the wound bed, a separate population of epithelial cells migrates to the top and these are derived from the basal cell layer where stem cells (i.e., undifferentiated epithelial cells) reside. Previously, it has been debated whether keratinocytes in a wound derive from the BMZ and ‘slide’ into the wound, or if the differentiated keratinocytes are the more migratory and ‘roll’ into the wound. Our data are consistent with both processes occurring, resulting in the inversion of the position of keratinocytes because the differentiated cells migrate to the bottom of the wound, while the stem cells (i.e., undifferentiated epithelial cells) migrate to the top. This makes sense because the bottom cells apoptose and die, so there is no need to have stem cells at the bottom, instead the undifferentiated epithelial cells go to the top where they are needed to produce the ever-replenishing epithelial layers.

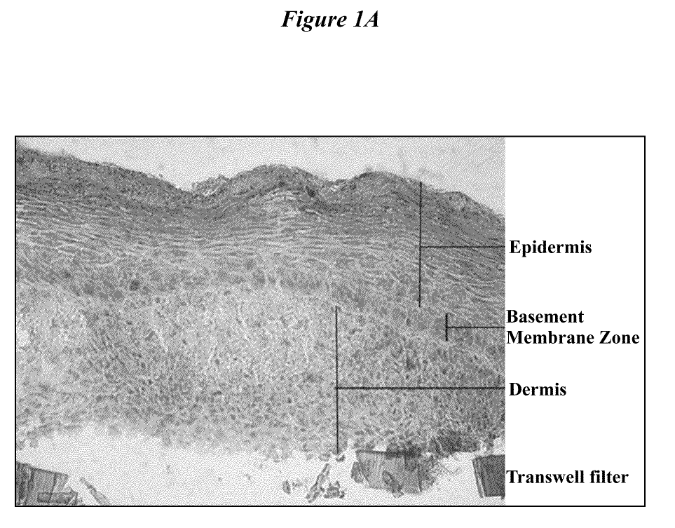

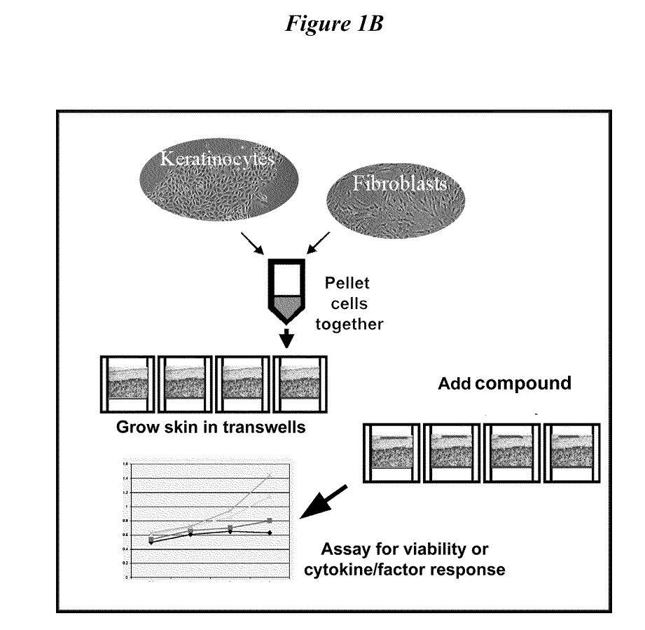

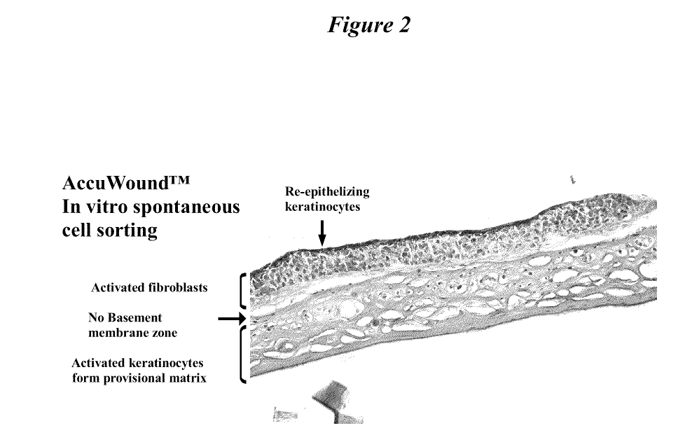

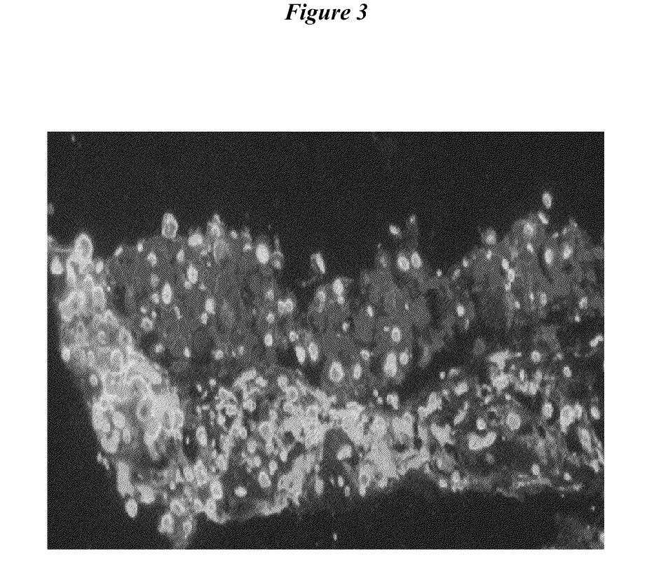

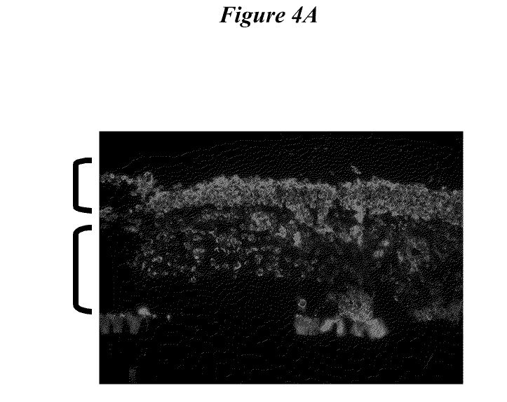

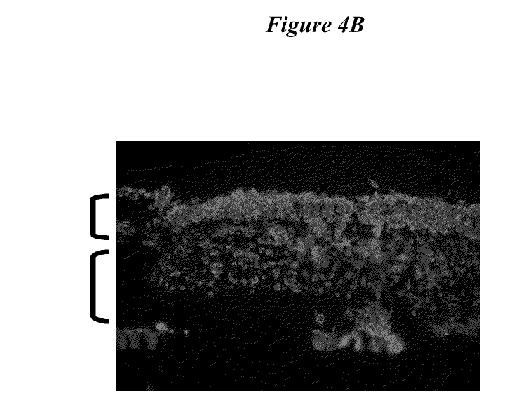

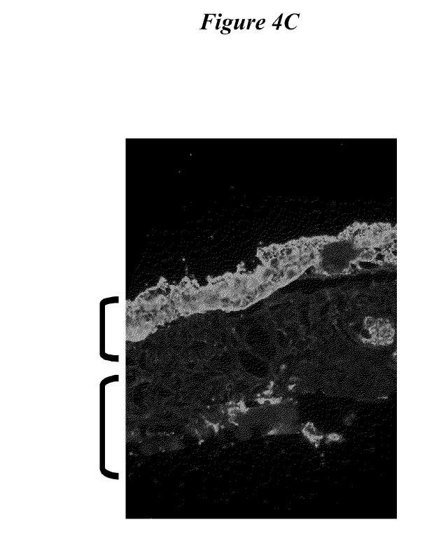

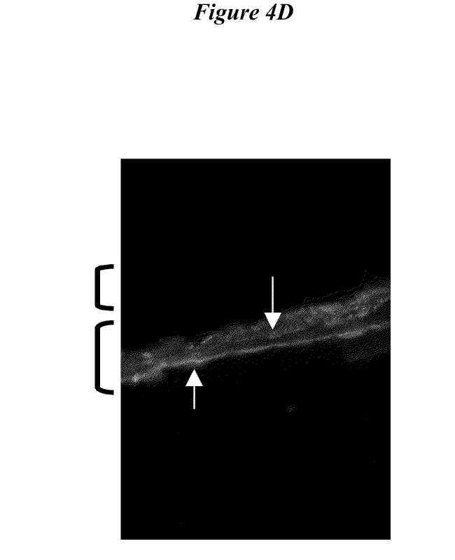

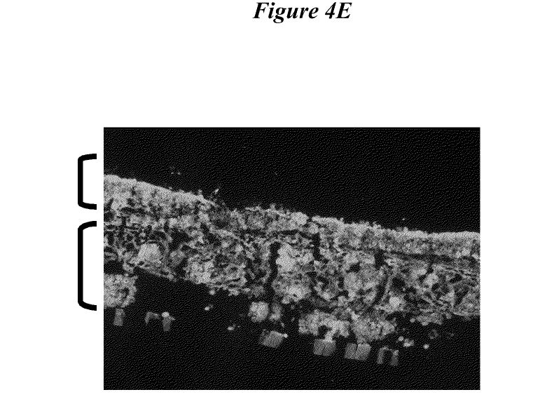

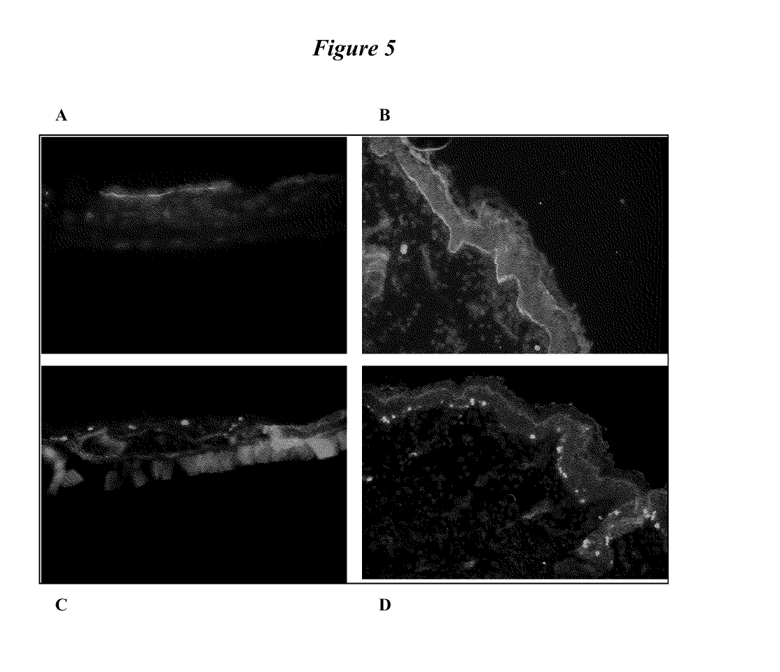

### DETAILED DESCRIPTION

1. Introduction

Accelerating and improving the quality of wound healing would have appreciable medical benefits for patients. Whereas the state of the art in using tissue cultured cells for grafting onto wounds has led to multiple treatment constructs that have some similarities to normal uninjured skin, the present application is based, in part, on the surprising discovery that greater success comes from using cultured cells to recreate a wound. This is because a key step in wound healing is the re-vascularization of the wound bed, where new blood vessels need to be formed to replace damaged vessels, and supply new replacement tissues with nutrients and oxygen. In an in situ wound epithelial cells are triggered to migrate into the wound bed and the present invention shows for the first time that that the differentiated epithelial cells apoptose and set up a spongy matrix or scaffold upon which invading cell types from the blood, for example, macrophages and endothelial cells, will attach. Accordingly, the present invention provides compositions composed of an epithelial cell derived scaffold allows re-vascularization of a wound bed and methods of use. By applying correctly responding epithelial cells, either alone, or within pre-made mesenchymal or epithelial/mesenchymal tissue constructs, practical use can be made of this epithelial cell created scaffold in facilitating the formation of a new microvasculature. By using the present in vitro generated apoptotic epithelial cell scaffolds, transplantation of tissues will quickly revascularize, leading to higher ‘take rates’ and persistence of grafts placed in wound beds.

The reason for the lack of success in getting in vitro made tissues to re-vascularize is that there has been a lack of understanding about what steps are required to allow re-vascularization to occur. Relatively little effort went into the pursuit of correctly modeling how epithelial/mesenchymal cell layers respond to wounding, in part because of a bias that normal uninjured looking skin should make the best grafting material. This was because of the reasonably good performance of donor skin either taken from elsewhere on the patient (autograft) or from a donor (allograft). The reason these grafts re-vascularize is that the donor tissues are vascularized to begin with, so the body only needs to reconnect the microvasculature at the graft site (angiogenesis). In contrast, in vitro produced grafts were never vascularized, and are even more dependent on wound healing mechanisms to create structures that allow re-vascularization (vasculogenesis). The scaffold made of apoptotic epithelial cells produced in vitro by the present invention is the structure needed to facilitate vascularization, and this is a structure created by the biological process of wound healing. The present invention is the recreation of an initial step in wound healing. Biological material recapitulating the wound is better designed to repair a wound than the use of material that was made to recapitulate normal uninjured tissue, as is currently the state of the art.

2. Cell Sorted Tissues

Cell sorted tissues of the present invention comprise a scaffold made of apoptotic epithelial cells. The cell sorted tissues can have one, two or three layers. In some embodiments, the cell sorted tissue is a scaffold or “spongy” matrix of apoptotic epithelial cells. In some embodiments, the cell sorted tissue is a tri-layer cell sorted tissue comprised of a bottom layer comprised of apoptotic epithelial cells, a middle layer comprised of mesenchymal cells, and a top layer comprised of undifferentiated epithelial cells.

In some embodiments, the epithelial cells and mesenchymal cells are present in about equal numbers. In some embodiments, the cell sorted tissues comprise a proportionately greater number of epithelial cells. For example, the ratio of epithelial cells to mesenchymal cell can be about 1:1, 2:1, 3:1, or more. In some embodiments, the mesenchymal layer is eliminated.

The cell sorted tissues of the present invention, generally do not form a basement membrane zone (BMZ). In some embodiments, a BMZ forms between the top layer undifferentiated epithelial cells and the middle layer mesenchymal cells. No BMZ forms between the bottom layer apoptotic epithelial cell matrix and the middle layer of mesenchymal cells. The mesenchymal middle cell layers partially migrate into the bottom epithelial cell scaffold after placement on the patient and in response to the invasion of blood cell types from the patient.

a. Bottom Layer—Apoptotic Epithelial Cell Scaffold

The cell sorted tissues of the present invention comprise an apoptotic epithelial cell scaffold. In tri-layered cell sorted tissues, the bottom layer is comprised of an apoptotic epithelial cell scaffold. The cells in the apoptotic epithelial cell matrix are differentiated epithelial cells, that do not divide, and the majority of which have undergone or are undergoing programmed cell death or apoptosis. At least about 90%, 91%, 92%, 93%, 94%, 95%, 96%, 97%, 98%, 98%, 99% or more of the differentiated epithelial cells in the bottom layer have undergone or are undergoing apoptosis. The remaining scaffold is comprised of cytoskeletal and extracellular matrix proteins and other cell debris from the apoptosed cells, and appears “spongy” and porous. The apoptotic epithelial cell scaffold generally has a thickness of about 10-20 cells, and can be more or less thick as desired.

The bottom layer is attached to a solid support, for example, a transwell membrane, a culture dish, a wound, a connective tissue matrix (e.g., fibronectin, collagen IV, laminin and mixtures thereof).

Epithelial cells from any source are suitable for inclusion in the cell sorted tissues. For example, the epithelial cells can be from skin (i.e., keratinocytes), kidney, colon, prostate, breast, heart, urogenital, vagina, lung, gut, blood vessels, etc. The epithelial cells are obtained from any passage number, although earlier passages tend to be healthier, for example 3 passages or less. In some embodiments, the source of the epithelial cells used for the generation of the cell sorted tissues is the host or patient to be treated.

b. Middle Layer—Mesenchymal Cells

The middle layer is comprised of mesenchymal cells. Mesenchymal cells from any source are suitable for inclusion in the cell sorted tissues. For example, the mesenchymal cells can be fibroblasts. In some embodiments, the source of the mesenchymal cells used for the generation of the cell sorted tissues is the host to be treated. The source of mesenchymal cells generally is chosen as a match for the epithelial cells. For example, the combination of epithelial cells and mesenchymal cells can derive both from the same organ, e.g., keratinocytes used with dermal fibroblasts, or endothelial cells used with smooth muscle cells, etc.

c. Top Layer—Undifferentiated Epithelial Cells

The top layer is comprised of undifferentiated epithelial cells that are still capable of dividing, and therefore form a replenishing layer of epithelial cells. These cells include stem cells and other undifferentiated cells, such as basal cells. Protocols that select for stem cells will yield greater number of cells residing in the top layers. These cells preferably are not exposed to serum. The undifferentiated epithelial cells in the top layer are generally from the same source as the differentiated epithelial cells in the bottom layer.

3. In Vitro Methods of Generating Cell Sorted Tissues

Basic in vitro methods for generating bi-layer cell sorted tissues are described in U.S. Pat. No. 6,815,202, the content of which is hereby incorporated herein by reference in its entirety for all purposes.

For in vitro preparation of the cell sorted tissues comprising a spongy apoptotic epithelial matrix, separately cultured epithelial cells and mesenchymal cells are subject to physical disruption. For example, they are harvested from attachment to the surface of a tissue culture substrate, e.g., a flask, a multiwell plate, e.g., by exposure to trypsin or by mechanical disruption. The epithelial cells are preferably cultured in media containing no serum (i.e., serum free media). In some embodiments, the epithelial cells can be cultured in media containing low levels of serum, e.g., 10% or less, for example, 5%, 2%, 1% serum. At this stage, the cells can be cultured in media containing about 200 μM or less of calcium, for example a concentration in the range of about 60-200 μM calcium.

The harvested epithelial cells and mesenchymal cells are mixed to form a homogenous mixture. As discussed above, an equal number of epithelial and mesenchymal cells can be mixed or a proportionately greater number of epithelial cells can be mixed with the mesenchymal cells. For example, the ratio of epithelial cells to mesenchymal cell can be about 1:1, 2:1, 3:1, or more. In some embodiments, the mesenchymal layer is eliminated and the epithelial cells are subject to culture media conditioned by mesenchymal cells. For example, factors produced by fibroblast cells, e.g, Keratinocyte growth factor (“KGF”) alone or in combination with fibroblast growth factor (“FGF”)-1, FGF-2, FGF-7, endothelial cell growth factor (“ECGF”), Insulin-like Growth Factor 2 (“IGF-2”), platelet-derived growth factor (“PDGF”)-α, PDGF-β, IL-6, IL-11, transforming growth factor (“TGF”)-β1, TGF-β3, and VEGF can be added to the culture medium in a concentration range of about 5-50 nanogram/mL each. The culture medium can be a commercially available serum free medium, for example, Epilife (Invitrogen Corp) and also contain insulin, selenium and transferrin (Invitrogen Corp) in addition to KGF, or the combination of factors, and may also contain vitamin C (e.g., at a concentration of about 50 mM).

The epithelial cells and mesenchymal cells are mixed to create a cell slurry having a density or concentration sufficient to allow cell contact and cell sorting. For example, the cells can be mixed at a density of about 5×10⁵ cells/μm² to about 10×10⁵ cells/μm², for example, about 5×10⁵ cells/μm², 6×10⁵ cells/μm², 7×10⁵ cells/μm², 8×10⁵ cells/μm², 9×10⁵ cells/μm², 10×10⁵ cells/μm² (e.g., about 3.5×10⁵ epithelial cells and about 3.5×10⁵ mesenchymal cells for a total of about 7×10⁵ cells in a surface area of 0.8 μm²). Stated another way, the cells can be mixed at a concentration of about 2000 cells/μL to about 10,000 cells/μL, for example, about 2000 cells/μL, about 3000 cells/μL, 4000 cells/μL, 4500 cells/μL, 5000 cells/μL, 6000 cells/μL, 7000 cells/μL, 8000 cells/μL, 9000 cells/μL or 10,000 cells/μL (e.g., about 3.5×10⁵ epithelial cells and about 3.5×10⁵ mesenchymal cells for a total of about 7×10⁵ cells in a volume of about 80 μL). The absolute number of cells is not critical. Fewer cells will spontaneously sort into a cell sorted tissue that is thinner. A greater number of cells will spontaneously sort into a cell sorted tissue that is thicker.

The homogenous mixture of cells is then plated on a transwell in sorting culture medium. The fibroblasts are briefly exposed to serum during the stopping of trypsinization, used to release the cells from their plates. The sorting culture medium is generally serum free (i.e., containing no serum). In some embodiments, the sorting culture medium contains low levels of serum, e.g., 10% or less, for example, 5%, 2%, 1% serum. The sorting culture medium can optionally contain added connective tissue components, e.g., fibronectin, collagen IV, laminin, and mixtures thereof. The sorting culture medium will generally have a calcium concentration of at least about 1.0 mM, for example, about 1.0 mM, 1.1 mM, or 1.2 mM.

The sorting of the cells into the cell sorted tissues of the invention takes place in the presence of an inflammatory cytokine, e.g., IL-1α, IL-1β, IL-6, IL-8, IL-12, IL-18, TNF-α, or a mixture thereof, at least during the first three days of sorting. The inflammatory cytokine can be present up to the entire cell sorting period, as desired. In some embodiments, the inflammatory cytokine is TNF-α. An inflammatory cytokine can be added to the sorting culture medium. The inflammatory cytokine can be added to the medium at a concentration in the range of e.g., about 5-50 nanogram/mL, for example, about 5 ng/mL, 10 ng/mL, 15 ng/mL, 20 ng/mL, 25 ng/mL, 30 ng/mL, 35 ng/mL, 40 ng/mL, 45 ng/mL or 50 ng/mL. Alternatively, the homogenous cell mixture is cultured under conditions such that the cells produce inflammatory cytokines themselves. This can be done by limiting the feeding media plus cells to about 80 μL contained in the top chamber and allowing the medium to expire (i.e., to become acidic) by not changing the medium for about a day following the initial plating (e.g., for 20 hours or more). The cells become stressed and produce inflammatory cytokines.

The homogenous cell mixture is cultured for a period of about 10 days (or more or fewer days, as needed). The differentiated and undifferentiated epithelial cells and mesenchymal cells spontaneously sort into a cell sorted tissue, the spongy scaffold of apoptosed epithelial cells forming at the bottom, a layer of mesenchymal cells forming in the middle, and dividing, undifferentiated epithelial cells migrate to the top.

4. Methods of Repairing Wounded Tissue

The layered cell sorted tissues described herein may be used as an artificial epithelial tissue for any mammal. Humans are the preferred mammal. However, the invention may be practiced with other mammals such as non-human primates and members of the bovine, ovine, porcine, equine, canine, and feline species, as well as rodents including mice, rats, and guinea pigs, and members of the lagomorph family including rabbits. The particular cell sorted tissue which is formed is generally dependent on the source of cells. For example, when human cells are used, a human layered cell sorted tissue is formed.

One benefit of cultivating the layered cell sorted tissues in vitro is that they are made under sterile conditions, and they are not contaminated by cells or factors contributed by a host animal to grow up a graft. Thus, in one embodiment, the layered cell sorted tissue made in tissue culture may be used as a skin graft that could be used on graft sites such as traumatic wounds and bum injury. The skin grafts may also be used to cover decubiti and ulcerations secondary to diabetes mellitus and venous stasis.

In performing methods of repairing wounded tissue, an apoptotic epithelial cell scaffold is first created according to the methods described herein. The apoptotic epithelial cell scaffold can be isolated or the bottom layer of a tri-layer cell sorted tissue. The apoptotic epithelial cell scaffold is then applied to the wounded tissue to promote healing and re-vascularization of the wound.

In another embodiment, reattachment of limbs is improved by covering surfaces containing vessels too small to reconnect with the apoptotic epithelial cell scaffold in addition to deliberately reattaching major vessels before suturing the limb. The scaffold creates a space ideally suited for the ingrowth of small vessels leading to improved reconnections and therefore improves overall vascularization. Similar applications of the apoptotic epithelial cell scaffold to surfaces of incisions, as required during surgical procedures, also accelerate wound healing at these sites. A related application entails the use of tissue engineered constructs, which may have been assembled using adult, embryonic, or transfected stem cells, for grafting back onto the patient.

In a further embodiment, the cell sorted tissues comprising the wound intermediate can be used in an in vitro assay to detect tissue responses to chemicals or drugs. Upon providing the cell sorted tissue comprising a wound intermediate the tissue is contacted with the chemical or drug of interest. The response is evaluated by comparing the effects on phenotype, gene expression patterns, and/or protein marker expression between the tissue before and after contact with the chemical or drug.

In a related aspect, the source of the cells used to assemble the in vitro tri-layer cell sorted tissues can be from patients known to suffer from diseases that effect wound healing.

In this case reconstruction of the wound in vitro is used as a tool for understanding the disease and provides a platform for conducting in vitro assays to detect tissue responses to chemicals or drugs that may be helpful in the treatment. Psoriasis is one common skin disease where in vitro cell sorted tissues derived from patient biopsies find use.

### EXAMPLES

The following examples are offered to illustrate, but not to limit the claimed invention.

### Example 1

An apoptotic epithelial cell scaffold was produced by including differentiated keratinocytes in a cell sorting reconstitution of skin. Equal numbers of cells (epithelial and mesenchymal) were individually cultured under serum free conditions. The fibroblasts are cultured in Dulbecco's Modified Eagle Medium (DMEM) containing IX insulin, transferring, selenium, and ethanolamine in proportion as specified by the manufacturer (InVitrogen Corp) in standard tissue culture treated flasks. The keratinocytes are cultured in Serum Free Medium or Epilife medium (InVitrogen Corp) in plasticware coated with collagen IV (Sigma Corp, or Becton Dickenson). Both cell types are harvested from tissue culture plasticware using 0.25% trypsin-EDTA and mixed together to create a slurry. Trypsinization is stopped by brief exposure to serum for fibroblasts and for keratinocytes was stopped with soybean trypsin inhibitor. Anopore (Whatman, Nunc) (although other membrane types also work) transwell inserts were used to deposit the slurries and the cultures were fed from the bottom and the top for the first 3-5 days. The diameter of the filter inserts in the transwells was 0.8 μm². 3.5×10⁵ mesenchymal cells and an equal number of epithelial cells were added together to make the slurry for each well. The method can be scaled up to contain proportionally equivalent numbers of cells for any increase in area of the filter used. The absolute cell number was not critical, so if fewer cells were used the resulting tissue made was just thinner. Inflammatory cytokines were present during the first 3 days of cell sorting for example TNF-α, but could also include IL-1α, IL-1β, IL-6, IL-8 and others included as present during inflammation. These inflammatory cytokines can be induced in the cells added by reducing the amount of growth medium to about 80 μl and allowing the medium to expire, turning yellow in the process, and not changing the medium for about 20 hours or more. Various types of culture medium can be tolerated, but DMEM works well supplemented with 1× insulin, transferring, selenium, ethanolamine in proportion as specified by the manufacturer (InVitrogen), and containing vitamin C at a concentration of 50 mM. By about 10 days in culture in a CO₂ incubator at 37° C. the scaffold will form at the bottom of the filter. The scaffold can be stored longer term and used by placing into a wound bed. Freeze drying, and other methods of stabilizing proteins for longer term storage, including refrigeration, help the stability of a product based on this technology.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated apoptotic epithelial cell scaffold.

2. The isolated epithelial cell scaffold of claim 1, wherein the scaffold is freeze-dried.

3. An isolated tri-layered cell sorted tissue comprised of
a) a bottom layer comprised of an apoptotic epithelial cell scaffold;
b) a middle layer comprised of mesenchymal cells; and
c) a top layer comprised of non-differentiated epithelial cells.

4. The tri-layered cell sorted tissue of claim 3, wherein the epithelial cells are keratinocytes.

5. The tri-layered cell sorted tissue of claim 3, wherein the mesenchymal cells are fibroblasts.

6. The tri-layered cell sorted tissue of claim 3, wherein the epithelial cells are from a tissue source selected from the group consisting of skin, kidney, colon, prostate, breast, heart, urogenital tissue, vagina, lung, gut and blood vessels.

7. The tri-layered cell sorted tissue of claim 3, wherein the mesenchymal cells are from a tissue source selected from the group consisting of skin, kidney, colon, prostate, breast, heart, urogenital tissue, vagina, lung, gut and blood vessels.

8. The tri-layered cell sorted tissue of claim 3, wherein the mesenchymal cells are endothelial cells or smooth muscle cells.

9. The tri-layered cell sorted tissue of claim 3, wherein the cell sorted tissue comprises about equal numbers of epithelial cells and mesenchymal cells.

10. The tri-layered cell sorted tissue of claim 3, wherein the cell sorted tissue does not comprise a basement membrane zone (BMZ).

11. An in vitro method of generating a tri-layered cell sorted tissue comprising a discrete bottom cell layer, a discrete middle cell layer and a discrete top cell layer comprising the steps of:
i) providing a homogenous mixture of cells, said mixture comprising epithelial cells and mesenchymal cells; and
ii) contacting said mixture with a membrane or a connective tissue component in the presence of an inflammatory cytokine and under conditions wherein said mixture spontaneously sorts into said bottom, middle and top discrete cell layers, wherein said discrete bottom layer comprises an apoptotic epithelial cell scaffold, said discrete middle layer comprises mesenchymal cells and said discrete top cell layer comprises non-differentiated epithelial cells.

12. The method of claim 11, wherein the epithelial cells are keratinocytes.

13. The method of claim 11, wherein the mesenchymal cells are fibroblasts.

14. The method of claim 11, wherein the epithelial cells are from a tissue source selected from the group consisting of skin, kidney, colon, prostate, breast, heart, urogenital tissue, vagina, lung, gut and blood vessels.

15. The method of claim 11, wherein the mesenchymal cells are from a tissue source selected from the group consisting of skin, kidney, colon, prostate, breast, heart, urogenital tissue, vagina, lung, gut and blood vessels.

16. The method of claim 11, wherein the mesenchymal cells are endothelial cells or smooth muscle cells.

17. The method of claim 11, wherein the homogenous mixture of cells comprises about equal numbers of epithelial cells and mesenchymal cells.

18. The method of claim 11, wherein the inflammatory cytokine is tumor necrosis factor alpha (TNF-α).

19. The method of claim 18, wherein the TNF-α is produced by the mixture of cells.

20. The method of claim 18, wherein the TNF-α is exogenously added to the mixture of cells.

21. The method of claim 11, wherein the conditions comprises at least 1.0 mM Ca²⁺.

22. The method of claim 11, further comprising the step of subjecting the epithelial cells and the mesenchymal cells to physical disruption before providing them in the homogenous mixture.

23. The method of claim 11, wherein the tri-layered cell sorted tissue does not comprise a basement membrane zone (BMZ).

24. A method of repairing wounded tissue, comprising
i) generating in vitro an isolated tri-layered cell sorted tissue comprised of
a) a bottom layer comprised of an apoptotic epithelial cell scaffold;
b) a middle layer comprised of mesenchymal cells; and
c) a top layer comprised of non-differentiated epithelial cells
ii) contacting the wound bed containing mesenchymal tissue and damaged blood vessels with epithelial tissue from the bottom layer of the trilayered cell sorted tissue, wherein the apoptotic epithelial cell scaffold promotes the infiltration of new blood vessels in the wound, thereby repairing the wounded tissue.

25. The method of claim 24, wherein the tissue is skin.

26. The method of claim 24, wherein the epithelial cells are keratinocytes.

27. The method of claim 24, wherein the mesenchymal cells are fibroblasts.

28. The method of claim 24, wherein the epithelial cells are from a tissue source selected from the group consisting of skin, kidney, colon, prostate, breast, heart, urogenital tissue, vagina, lung, gut and blood vessels.

29. The method of claim 24, wherein the mesenchymal cells are from a tissue source selected from the group consisting of skin, kidney, colon, prostate, breast, heart, urogenital tissue, vagina, lung, gut and blood vessels.

30. The method of claim 24, wherein the mesenchymal cells are endothelial cells or smooth muscle cells.
